(19) United States
(12) Patent Application Publication
(10) Pub. No.: US 2011/0200691 A1
(43) Pub. Date: Aug. 18, 2011

(54) Medicine for Filterable Virus Infections and Its Processing Method

(76) Inventor: Shek Lam Chan, Kowloon (HK)

(21) Appl. No.: 12/439,545

(22) PCT Filed: Jan. 22, 2008
(86) PCT No.: PCT/CN2008/000159

§ 371 Date: Sep. 30, 2010

(51) Int. Cl. A61K 36/752; A61P 31/12; A61P 31/20; A61P 31/22; A61P 31/14

(52) U.S. Cl. 424/736

(57) ABSTRACT

A medicine for filterable virus infections and its processing method, based on the theory of the traditional Chinese medicine. The main material used is pericarpium citri reticulatae (hereinafter referred to as “PCR”) ground into powder after being stir-baking and burning to carbon black. After mixed with certain primary agent, the medicine in the present invention may be made into medical ointment for external use or powder or pill for oral medication, which is eutherapeutic to viral infections, such as lip boil, human papilloma virus (HPV), herpes zoster, genital herpes, chickenpox, influenza and virus diarrhea.

### BACKGROUND OF THE INVENTION

The present invention is about a Chinese herbal medicine and its processing method, and relates to a medicine made from pericarpium citri reticulatae (“PCR”) and its processing method.

PCR, in prescription, is usually called orange peel.

PCR is the skin of ripe orange. According to the Chinese pharmacopoeia, it is warm in nature, bitter and pungent in flavor, plays the functions of spleen-nourishing, qi-regulating, dampness-removing and phlegm-resolving. Pharmacologically, it is anti-inflammatory, antiulcer, cholagogue, antispetic, blood pressure ascending, cordial and phlegm removing. In addition, it accelerates the secretion of digestive juice and eliminates the constipation causing gas from the intestine. Traditionally, it has always been accompanied with other herbal medicines in medical use, and widely used by folks for preserved fruit, or as an ingredient in pastries and cakes, confiture and drinks. Recent years, use of PCR has been more and more popular, and more than 20 patent applications in China have stated its uses for orange peel tea, milk products, liquors for health-care, pastries and cakes, or confiture. With the application of modern techniques to the produce of and analysis to the Chinese herbal medicine, the chemical composition and pharmacology of extracts from the PCR are more and more clearly known to us. The chemical composition in the PCR is mainly consisted of d-limonene, citral, hesperidin, tangeretin, neobiletin and citromitin etc. And 2-pinene and terpinene are contained in the volatile oil of PCR.

Through compounding the extracts from the PCR and those from other Chinese herbal medicine, new functions may be expected from the mix, e.g. According to the China patent with the patent number of 200510034709.4, medicines for anti-myocardial ischaemia, anti-thrombopalstin, blood-fat reducing, blood pressure regulation and anti-atherosclerosis may be prepared after mixing the extracts from the PCR and the ginger. In a word, the application of PCR is more and more widely.

At present, there is no record or publication on the use of processed PCR to treat filterable viral infection.

### BRIEF SUMMARY OF THE INVENTION

The present invention aims to offer a medicine with PCR as the raw material to treat filterable virus infections diseases, and to illuminate the processing method for the said medicine, which may be directly used on the part in disease, functions eutherapeuticly to the filterable virus infections diseases. Advantages of the medicine lie in that it may quickly set the patient recovered with low recurring risk, and usually it may completely cure the disease with extremely low side-effect, and it is harmless.

The technical solutions to realize the object of the present invention are listed as follows:

The said medicine uses PCR as the raw material, which is ground into powder after being stir-baking and burning to carbon black.

The process of the said processing method is as follows: Firstly, put the dry PCR into a wok, then heat the wok and meanwhile stir fry the PCR. Add kindling into the wok after all the PCR becomes black by heating and stir-fry. And then extinguish the fire after the PCR is burnt to carbon black. Cool it and grind it into powder.

The said processing method may be realized in accordance the following steps: Put 1.8 Kg of dry PCR into a wok which has a diameter of 50 cm, with the thickness of 1.32 mm in the bottom. Then, heat the wok and meanwhile stir fry the PCR for about 35 to 45 minutes. And the PCR will become black when the temperature in the wok amounts to between 160° C. and 200° C. And then, add kindling into the wok to burn the raw material PCR, and the temperature in the wok for the time being is more than 330° C. Keep the PCR burned for 2 to 4 minutes until the temperature rises to between 410° C. to 450° C. Then cover the wok to extinguish the fire, and all the PCR will have been burnt to carbon black. Take it out after it cools, and then grind it into powder through mesh of 150.

The said processing method may also be realized in accordance the following steps: Put 20 kg of dry PCR into a rotor electric wok which has a diameter of 70 cm and a length of 100 cm, with the thickness of 2.0 mm in the wok shell. Power on the wok and meanwhile stir fry the PCR for about 45 to 55 minutes. And the PCR will become black when the temperature in the wok amounts to between 160° C. and 200° C. Then cut off the electricity supply to the wok, and open the cap of the wok. Pour the black PCR into a pig iron wok with thick shell or a container, and add kindling into the iron wok to burn the raw material PCR, and the temperature in the wok for the time being is more than 350° C. Keep the PCR burned for 2 to 3 minutes until the temperature rises to between 410° C. to 450° C. Then cover the iron wok to extinguish the fire, and all the PCR will have been burnt to carbon black. Take it out after it cools, and then grind it into powder through mesh of 150.

The processing time for the said processing method should be determined according to the diameter and thickness of the wok used. The stir-fry duration should be determined in accordance with the change of the raw material PCR being stir-fried. The key point is not to burn the PCR in the wok until all of it totally becomes black, and not to extinguish the fire until it is burnt to carbon black. In addition, grind the carbon black PCR into powder through mesh of 100-250 after it cools.

After processed with the said processing method, the medicine in the present invention will appear in the form of black powder obtained by stir-fry. The medicine in the present invention wears a single shape and is easy to use. More than ten years clinical trials indicate that it is highly effective to treat the filterable virus infections. And it is extremely effective for treating skin ulcers caused by herpes virus, herpes zoster virus, filterable virus, influenza virus, chickenpox virus, HPV, and lip boil virus. And it features for short course of treatment, low recurring risk, high completely curing effect, being easily dissolved in water and harmless, and no side-effect has been discovered until now.

The medicine of the present invention improves functioning of the monomacrophage system. It can regulate cellular and humoral immunity. It is anti-ulcus and anti-inflammatory. It can enhance breeding of immunological cells and phagocytosis of macrophage.

It is still unclear why using the PCR after it has been burnt to carbon black and ground into powder could yield such effects.

The PCR is used as the raw material for the medicine in the present invention. In processing, a dry PCR with the water content of or below 4% is adopted. Firstly, put the dry PCR into the wok whose size and pattern may be different as the quantity of the raw material and the processing condition are different. For small sized wok, the manual stir-fry, just like the way to fry tea leaves, may be employed. But for industrial production in a large scale, electric wok such as rotating circular electric wok, rotor electric wok or electric wok in other forms should be used so as to control the temperature and to standardize the stir-fry process. In the processes of heating and stir-fry, the color change of the PCR should be carefully observed so as to control the stir-fry duration. When the PCR becomes black, kindling should be thrown into the wok to burn the PCR until it takes on an irregular shape in carbon black color. Then extinguish the fire, and naturally cool the PCR under room temperature. After it completely cools, the PCR in carbon black color should be ground into powder through mesh of 100-250. The medicine may be made into medical ointment for external use or powder or pill for oral medication.

### Embodiment I

Put 1.8 kg of dry PCR into a wok which has a diameter of 50 cm, with the thickness of 1.32 mm in the bottom. Then, heat the wok and meanwhile stir fry the PCR for about 35 to 45 minutes. And the PCR will become black when the temperature in the wok amounts to between 160° C. and 200° C. And then, add kindling into the wok to burn the raw material PCR, and the temperature in the wok for the time being is more than 330° C. Keep the PCR burned for 2 to 4 minutes until the temperature rises to between 410° C. to 450° C. Then cover the wok to extinguish the fire, and all the PCR will have been burnt to carbon black. Take it out after it cools, and then grind it into powder through mesh of 150.

### Embodiment II

Put 20 kg of dry PCR into a rotor electric wok which has a diameter of 70 cm and a length of 100 cm, with the thickness of 2.0 mm in the wok shell. Power on the wok and meanwhile stir fry the PCR for about 45 to 55 minutes. And the PCR will become black when the temperature in the wok amounts to between 160° C. and 200° C. Then cut off the electricity supply to the wok, and open the cap of the wok. Pour the black PCR into a pig iron wok with thick shell or a container, and add kindling into

1-5. (canceled)

6. A medicine for treating filterable virus infections, wherein the said medicine uses PCR as raw material, which will be ground into powder after being stir-fried and burnt into carbon black.

7. The medicine for treating filterable virus infections as in claim 6, wherein the said powder may be mixed with certain primary agent to form medical ointment, and the said powder weighs 10% to 20% of the medical ointment.

8. The medicine for treating filterable virus infections as in claim 7, wherein the said certain primary agent may be made from a single or mixture of liquid paraffin, waxy paraffin and soft paraffin in arbitrary proportion.

9. A processing method for the medicine for treating filterable virus infections as claimed in claim 6, wherein process of the said processing method is as follows: firstly, put dry PCR into a wok, then heat the wok and meanwhile stir fry the PCR; add kindling into the wok after all the PCR becomes black by heating and stir-fry; and then extinguish fire after the PCR is burnt to carbon black; cool it and grind it into powder.

10. The processing method for the medicine for treating filterable virus infections as in claim 9, wherein processing time for the said processing method should be determined according to diameter and thickness of the wok used; stir-fry duration should be determined in accordance with change of the raw material PCR to be stir-fried; key point is not to burn the PCR in the wok until all of it totally becomes black, and not to extinguish fire until it is burnt to carbon black; in addition, grind the carbon black PCR into powder filtered through mesh of 80 to 250 after it cools.

11. The processing method for the medicine for treating filterable virus infections as in claim 10, wherein the said processing method may be realized in accordance to the following steps: put 1.8 kg of dry PCR into a wok which has a diameter of 50 cm, with a thickness of 1.32 mm in the bottom; then, heat the wok and meanwhile stir fry the PCR for about 35 to 45 minutes; and the PCR will become black when temperature in the wok amounts to between 160° C. and 200° C.; and then, add kindling into the wok to burn the raw material PCR, and temperature in the wok for the time being is more than 330° C.; keep the PCR burned for 2 to 4 minutes until temperature rises to between 410° C. to 450° C.; then cover the wok to extinguish fire, and all the PCR will have been burnt to carbon black; take it out after it cools, and then grind it into powder filtered through mesh of 150.

12. The processing method for the medicine for treating filterable virus infections as in claim 10, wherein the said processing method may be realized in accordance to the following steps: put 20 kg of dry PCR into a rotor electric wok which has a diameter of 70 cm and a length of 100 cm, with a thickness of 2.0 mm in wok shell; power on the wok and meanwhile stir fry the PCR for about 45 to 55 minutes; and the PCR will become black when temperature in the wok amounts to between 160° C. and 200° C.; then cut off electricity supply to the wok, and open cap of the wok; pour the black PCR into a pig iron wok with thick shell or a container, and add kindling into the iron wok to burn the raw material PCR, and temperature in the wok for the time being is more than 350° C.; keep the PCR burned for 2 to 3 minutes until temperature rises to between 410° C. to 450° C.; then cover the iron wok to extinguish fire, and all the PCR will have been burnt to carbon black; take it out after it cools, and then grind it into powder filtered through mesh of 150.

13. The processing method for the medicine for treating filterable virus infections as in claim 11, wherein the said fire extinguishing means that directly cover the wok to make the fire go out after the PCR completely burnt to carbon black.

14. The processing method for the medicine for treating filterable virus infections as in claim 12, wherein the said fire extinguishing means that directly cover the wok to make the fire go out after the PCR completely burnt to carbon black.

15. Application of the said medicine recited in claim 6 to the treatment for wart virus HPV, lip boil, chickenpox, berpes zoster and genitalia herpes, wherein the medicine or the medical ointment made from the medicine may be directly applied to the sore, 2 to 5 times a day.

16. The application of the said medicine to the treatment for wart virus HPV, lip boil, chickenpox, berpes zoster and genitalia herpes as in claim 15, wherein the medicine should be directly applied to the festering sore, i.e. the medicine shall be applied only after the sore has festered.

17. Application of the said medicine recited in claim 6 to the treatment for influenza and virus diarrhea, wherein 1 g to 2 g of the medicine should be taken after adequately blending it into water of 200 ml; take it 3 to 4 times a day.
